(19) United States
(12) Patent Application Publication
(10) Pub. No.: US 2026/0284431 A1
(43) Pub. Date: Sep. 24, 2026

(54) Methods of Treating and Preventing Cancer with Alternating Electric Fields, Radioactive Particles, and Systemic Therapy

(71) Applicant: Novocure GmbH, Baar (CH)

(72) Inventor: William DOYLE, Malvern, PA (US)

(73) Assignee: Novocure GmbH, Baar (CH)

(21) Appl. No.: 19/676,273

(22) Filed: May 13, 2026

Related U.S. Application Data
(63) Continuation of application No. 17/837,638, filed on Jun. 10, 2022, now abandoned.
(60) Provisional application No. 63/210,173, filed on Jun. 14, 2021.

(51) Int. Cl. A61N 5/10; A61K 31/44; A61K 31/7072; A61K 51/12; A61N 1/40; A61P 35/00

(52) U.S. Cl. CPC A61N 5/1002; A61K 31/44; A61K 31/7072; A61K 51/1251; A61N 1/40; A61P 35/00; A61N 2005/1024

(57) ABSTRACT

Methods of treating and preventing cancer are provided. In some instances, the method comprises delivering radioactive particles to an organ of the subject, wherein the organ contains a tumor, applying alternating electric fields to the organ at a frequency of 50 kHz to 10 MHz (e.g., 50 kHz to 1 MHz or 80 to 500 kHz), and administering systemic cancer therapy to the subject.

### CROSS REFERENCE TO RELATED APPLICATIONS

This Application is a continuation of U.S. Ser. No. 17/837,638, filed Jun. 10, 2022, which claims the benefit of U.S. Provisional Application 63/210,173, filed Jun. 14, 2021, each of which is incorporated herein by reference in its entirety.

### BACKGROUND

Colorectal cancer (CRC) is the third most commonly diagnosed cancer and a leading cause of cancer-related mortality¹. The liver is the most common and often predominant site of metastases, as the site of portal venous drainage of the colon and rectum, occurring in up to 60% of CRC patients during the course of their disease^{2, 3}. Oligometastatic colorectal cancer confined to the liver represents an intermediate state in the evolution of metastatic capacity that opens the opportunity for local interventions. Locoregional therapy, such as transarterial chemoembolization (TACE) or radioembolization (TARE) are considered therapeutic options for selected patients. These modalities are aimed to the confined area of the tumor(s), thus sparing the toxicity of systemic treatment.

TARE, also known as selective internal radiation therapy (SIRT) is a form of intra-arterial brachytherapy where microspheres loaded with the beta particle emitter 90Y (Yttrium-90) are delivered via catheters placed into tumor-supplying hepatic arteries. Currently, there are two commercially available microspheres: THERASPHERE® using glass microspheres and SIR-Spheres® using resin spheres. In the United States, only the resin microsphere is approved by the FDA for use in CLM (colorectal liver metastases) patients, therefore the bulk of the published literature on TARE in CLM is with the resin microspheres, either as monotherapy or in combination with chemotherapy⁴.

Initial clinical reports of TARE treatment of patients with unresectable liver-only or liver dominant CRC were in the chemotherapy refractory settings. This means that in most cases patients had failed first and second line of standard systemic therapy, which typically means prior exposure to 5-FU, oxaliplatin and irinotecan^{4, 5}. A great majority of the prospective and retrospective published studies on TARE for unresectable CLM are in this setting (summarized in Wang et al., 2019)⁴.

The liver is the most common site of metastasis for colorectal cancer patients because of its central role in portal circulation¹⁵. Incidence levels as high as 35% at first medical consultation have been shown using computed topography (CT) scans¹⁵. Among 1,450,000 patients with a recent diagnosis of colorectal cancer 30,000 to 40,000 will develop synchronic metastasis or metachronous metastasis in the liver¹⁵. Despite improvements in detection and prognosis of colorectal cancer, metastasis of colorectal cancer to the liver reduces patient survival¹⁵. In addition, due to the location and size of the liver and the complication of additional hepatic disease, 85% of patients are not eligible for surgery¹⁵.

Tumor Treating Fields (TTFields) are an effective anti-neoplastic treatment that involves applying low intensity, intermediate frequency (e.g., 80-500 kHz), alternating electric fields (AEFs) to a target region.

In the in vivo context, TTFields therapy can be delivered using a wearable and portable device (Optune®). The delivery system includes an electric field generator, four adhesive patches (non-invasive, insulated transducer arrays), rechargeable batteries and a carrying case. The transducer arrays are applied to the skin and are connected to the device and battery. The therapy is designed to be worn for as many hours as possible throughout the day and night. In the preclinical setting, TTFields can be applied in vitro using, for example, the Inovitro™ TTFields lab bench system. Inovitro™ includes a TTFields generator and base plate containing 8 ceramic dishes per plate. Cells are plated on cover slips placed inside each dish. TTFields are applied using two perpendicular pairs of transducer arrays insulated by a high dielectric constant ceramic in each dish. In both the in vivo and in vitro contexts, the orientation of the TTFields is switched 90° every 1 second, thus covering different orientation axes of cell divisions.

### SUMMARY OF THE INVENTION

One aspect is directed to a first method of treating cancer in a subject diagnosed with or suspected of having cancer. The first method comprises delivering radioactive particles to an organ of the subject, wherein the organ contains at least one cancer cell, applying alternating electric fields to the organ at a frequency of 50 kHz to 10 MHz, and administering systemic cancer therapy to the subject.

Another aspect is directed to a second method of preventing recurrence of cancer in a subject that has been previously treated for cancer comprising applying alternating electric fields to an organ of the subject at a frequency of 50 kHz to 10 MHz for a period of at least three months.

A further aspect is directed to a third method of treating colorectal cancer that has metastasized to a liver in a subject. The third method comprises delivering radioactive particles to a liver of the subject, wherein the liver contains metastatic colorectal cancer cells; applying alternating electric fields to the organ at a frequency of 50 kHz to 10 MHz; and administering systemic cancer therapy to the subject.

### BRIEF DESCRIPTION OF THE DRAWINGS

FIG. 1 shows an exemplary study scheme for a method of treating a subject having metastatic cancer.

### DESCRIPTION

One aspect is directed to a first method of treating cancer in a subject diagnosed with or suspected of having cancer. The first method comprises delivering radioactive particles to an organ of the subject, wherein the organ contains at least one cancer cell, applying alternating electric fields to the organ at a frequency of 50 kHz to 10 MHz (e.g., 50 kHz to 1 MHz or 80 to 500 kHz), and administering systemic cancer therapy to the subject. In some instances, the cancer is a primary cancer. In some instances, the cancer is a metastatic cancer.

In some instances of the first method, the alternating electric field has a frequency between 100 and 300 kHz. In some instances, at least a portion of the applying step is performed simultaneously with at least a portion of the delivering step.

The term “administering systemic cancer therapy” refers to providing the systemic cancer therapy (e.g., chemotherapeutic agent) to a patient by a healthcare professional or the patient through any suitable and accepted route of administration (e.g., oral, intravenous, parenteral, topical etc.) as approved on the product label by a regulatory authority, under the care of a healthcare professional, or as part of an approved clinical trial. Prescribing a checkpoint inhibitor can also be “administering” a checkpoint inhibitor.

In some instances of the first method, the radioactive particles are selected from the group consisting of resin-based microspheres and glass-based microspheres.

Examples of microspheres include THERASPHERE® using glass microspheres and SIR-Spheres® using resin spheres.

In some instances of the first method, the radioactive particles are glass-based microspheres. In some instances of the first method, radioactive particles are glass-based microspheres comprising Yttrium 90. In some instances of the first method, the microspheres comprise Cesium-131, Gold-198, Iridium-192, Iodine-125, Indium 111 (In-111) or Gallium-68 (Ga-68). See, e.g., U.S. Published Patent Application 20160331854.

In some instances of the first method, the radioactive particles are delivered to the organ. For example, the radioactive particles can be delivered by introducing a catheter containing the particles through an artery (e.g., hepatic artery) to a target organ (e.g., liver). In another aspect, the radioactive particles can be delivered to the at least one cancer cell in the organ. The cancer cells or a tumor containing the cancer cells can be identified by, for example, magnetic resonance imaging (MRI), CT or another imaging method. Radioactive particles can be delivered, for example, under CT-guided fluoroscopy to through a catheter to the tumor site.

In some instances of the first method, the frequency of the alternating electric fields is from 120 kHz to 180 kHz. In some instances, the frequency of the alternating electric fields is or is about 150 kHz.

In some instances of the first method, the alternating electric fields are applied for 30 minutes to 24 hours or longer. In some instances of the first method, alternating electric fields are applied for at least 3 hours. In some instances of the first method, the alternating electric fields are applied for at least 18 hours. Alternating electric fields can be applied continuously or discontinuously.

The term “continuously” refers to applying alternating electric fields for a substantially constant period of time. Continuous application of alternating electric fields can occur even if the application is discontinued for a short period of time (e.g., seconds) in order to position equipment appropriately, or if there is a brief disruption of power.

The term “discontinuously” refers to applying alternating electric fields for a period of time with a periodic break or disruption for seconds, minutes, an hour, days or more. In this aspect, a patient could apply alternating electric fields for a period of time (e.g., 1, 2, 3, 4, 8, 24, 48, 72 hours) with a 15 minute, 30 minute, 45 minute, 1 hour period without applying the alternating electric field. In another aspect, the patient could apply the alternating field continuously while sleeping and discontinuously while awake. In a further aspect, the patient can apply the alternating electric field continuously except during mealtime or during a social event.

In some instances of the first method, the organ is located in an abdomen of the subject (e.g., liver, pancreas, bile duct, and spleen). In some instances of the first method, the organ is located in a head of the subject (e.g., brain). In some instances of the first method, the organ is a liver.

In some instances of the first method, the alternating electric fields are applied prior to or during the delivering of the radioactive particles. In some instances of the first method, the alternating electric fields are applied after or during the administering of the systemic cancer therapy.

In some instances of the first method, the systemic cancer therapy comprises administering a chemotherapeutic agent to the subject. For example, the chemotherapeutic agent can be administered by injection, orally, or topically. In some instances of the first method, more than one chemotherapeutic agent is administered.

In some instances of the first method, the chemotherapeutic agent or agents are determined to be the “standard of care” for a particular type of primary or metastatic cancer. In some instances of the first method, the chemotherapeutic agent or agents are determined to be the “standard of care” for liver cancer (e.g., hepatocellular carcinoma).

In some instances of the first method, the chemotherapeutic agent is selected from one or more of regorafenib and trifluridine.

Another aspect is directed to a second method of preventing recurrence of cancer in a subject that has been previously treated for cancer comprising applying alternating electric fields to an organ of the subject at a frequency of 50 kHz to 10 MHz (e.g., 50 kHz to 1 MHz, or 80 to 500 kHz) for a period of at least three months.

The term “a subject that has been previously treated for cancer” refers to a subject that has been previously treated for cancer (e.g., primary or metastatic) and is at risk of developing a recurrence of the cancer. In some instances, the subject has been previously treated for cancer and does not exhibit any symptoms or diagnostic markers of having cancer following treatment.

In some instances of the second method, the alternating electric field has a frequency between 100 and 300 kHz. In some instances of the second method, at least a portion of the applying step is performed simultaneously with at least a portion of the delivering step.

In some instances of the second method, radioactive particles can be delivered to the organ. The organ can contain at least one cancer cell. A tumor containing the at least one cancer cell can be identified by, for example, magnetic resonance imaging (MRI), CT or another imaging method. Radioactive particles can be delivered, for example, under MRI, ultrasound, or CT-guided fluoroscopy to the tumor site.

In some instances of the second method, the frequency of the alternating electric fields is from 120 kHz to 180 kHz. In some instances of the second method, the frequency of the alternating electric fields is or is about 150 kHz.

Alternating electric fields can be applied continuously or discontinuously.

In some instances of the second method, the organ is located in an abdomen of the subject (e.g., liver, pancreas, bile duct, and spleen). In some instances of the second method, the organ is located in a head of the subject (e.g., brain). In some instances of the second method, the organ is a liver.

In some instances of the second method, the alternating electric fields are applied prior to or during the delivering of the radioactive particles. In some instances of the second method, the alternating electric fields are applied after or during the administering of the systemic cancer therapy.

In some instances of the second method, the systemic cancer therapy comprises administering a chemotherapeutic agent to the subject. For example, the chemotherapeutic agent can be administered by injection, orally, or topically. In some instances of the second method, more than one chemotherapeutic agent is administered. In some instances of the second method, the chemotherapeutic agent or agents are determined to be the “standard of care” for a particular type of primary or metastatic cancer. In some instances of the second method, the chemotherapeutic agent or agents are determined to be the “standard of care” for liver cancer (e.g., hepatocellular carcinoma).

In some instances of the second method, the chemotherapeutic agent is selected from one or more of regorafenib and trifluridine.

A further aspect is directed to a third method of treating colorectal cancer that has metastasized to a liver in a subject. The third method comprises delivering radioactive particles to the liver of the subject, wherein the liver contains metastatic colorectal cancer cells; applying alternating electric fields to the organ at a frequency of 50 kHz to 10 MHz (e.g., 50 kHz to 1 MHz or 80 to 500 kHz); and administering systemic cancer therapy to the subject.

In some instances of the third method, the frequency of the alternating electric fields is from 120 kHz to 180 kHz. In some instances of the third method, the frequency of the alternating electric fields is or is about 150 kHz.

In some instances of the third method, the alternating electric fields are applied for at least 18 hours.

In some instances of the third method, the radioactive particles are selected from the group consisting of resin-based microspheres and glass-based microspheres. In some instances of the third method, the radioactive particles are glass-based microspheres. In some instances of the third method, the radioactive particles comprise Yttrium 90.

Examples of microspheres include THERASPHERE® using glass microspheres and SIR-Spheres® using resin spheres. In some instances of the third method, the radioactive particles are glass-based microspheres. In some instances of the third method, radioactive particles are glass-based microspheres comprising Yttrium 90. In some instances of the third method, the microspheres comprise Cesium-131, Gold-198, Iridium-192, Iodine-125, Indium 111 (In-111) or Gallium-68 (Ga-68). See, e.g., U.S. Published Patent Application 20160331854.

The radioactive particles can be delivered by introducing a catheter containing the particles through the hepatic artery to the liver. The tumor can be identified by, for example, magnetic resonance imaging (MRI), CT or another imaging method. Radioactive particles can be delivered, for example, under MRI, ultrasound, or CT-guided fluoroscopy to through a catheter to the metastatic colorectal tumor site.

In some instances of the third method, the systemic cancer therapy comprises administering a chemotherapeutic agent to the subject. For example, the chemotherapeutic agent can be administered by injection, orally, or topically. In some instances of the third method, more than one chemotherapeutic agent is administered.

In some instances of the third method, the chemotherapeutic agent or agents are determined to be the “standard of care” for a particular type of primary or metastatic cancer. In some instances of the second method, the chemotherapeutic agent or agents are determined to be the “standard of care” for liver cancer (e.g., hepatocellular carcinoma).

In some instances of the third method, the chemotherapeutic agent is selected from one or more of regorafenib and trifluridine.

In some instances of the methods described herein, the intensity of the alternating electric fields is 0.1 to 20 V/cm (RMS), 0.5 to 10 V/cm, 1 to 10 V/cm, 1.0 to 4 V/cm, or 1.0 to 2.5 V/cm (RMS) for at least a portion of the region to which the AEF is applied.

The non-limiting example below illustrates how to make and use aspects described herein and provide additional supporting data, with reference to the Figure, for the embodiments and aspects described herein, including modifications and alternations. Without being bound by any theories or hypotheses, the examples may include possible explanations for the described data. Accordingly, it is intended that the present invention not be limited to the examples provided below, but that it has the full scope defined by the language of the claims listed below, and equivalents thereof.

### EXAMPLE

A pilot, open-label, randomized controlled Study of Tumor Treating Fields (TTFields, 150 kHz) concomitant with Yttrium 90 glass microspheres (THERASPHERE®) and systemic treatment in patients with colorectal cancer liver metastases.
Indication—Recurrent colorectal cancer liver metastases

Study primary hypotheses: It is hypothesized that the addition of TTFields to radioembolization (RAE) using Yttrium 90 glass microspheres (THERASPHERE®) and systemic therapy in patients with colorectal cancer liver metastases will improve the time to liver metastases progression with an acceptable safety profile.

Study Population: Patients with refractory colorectal cancer liver-only or liver-dominant metastases which are not amenable to surgical resection and are candidates for localized treatment (radioembolization).

### Objectives

### Primary Objective

To evaluate the time to progression of liver metastases (TTLP)—evaluated by investigator according to RECIST 1.1 in patients with colorectal cancer liver metastases treated with TTFields in addition to Yttrium 90 glass microspheres and systemic therapy.

### Secondary Objectives

(1) To evaluate the time to progression (TTP) by RECIST 1.1 in patients with colorectal cancer liver metastases treated with TTFields in addition to Yttrium 90 glass microspheres and systemic therapy.
(2) To evaluate the safety and tolerability profile of TTFields in addition to Yttrium 90 glass microspheres and systemic therapy in patients with colorectal cancer liver metastases.
(3) To evaluate the overall survival (OS) of patients with colorectal cancer liver metastases treated with TTFields in addition to Yttrium 90 glass microspheres and systemic therapy.
(4) To evaluate the progression-free survival (PFS) by RECIST v1.1 in patients with colorectal cancer liver metastases treated with TTFields in addition to Yttrium 90 glass microspheres and systemic therapy.
(5) To evaluate the objective response rate (ORR) by RECIST v1.1 in patients with colorectal cancer liver metastases treated with TTFields in addition to Yttrium 90 glass microspheres and systemic therapy.
(6) To evaluate the disease control rate (DCR) at 6 months by RECIST 1.1 in patients with colorectal cancer liver metastases treated with TTFields in addition to Yttrium 90 glass microspheres and systemic therapy.

### Exploratory Objectives

To explore biomarkers correlating with response to treatment.

### Randomization and Stratification

Randomization will be 1:1 to either TTFields+Yttrium 90 glass microspheres (THERASPHERE®)+systemic therapy vs. Yttrium 90 glass microspheres (THERASPHERE®)+systemic therapy.

Stratification will be based on:
(1) Institution
(2) Type of progression (synchronous=progression while on chemotherapy or ≤6 months post chemotherapy vs. metachronous=progression occurring more than 6 months after cessation of chemotherapy)
(3) Presence or absence of extrahepatic disease

### Treatment Groups

### Treatment Arm I (Investigational Arm)

TTFields at 150 kHz to the liver, continuous for at least 18 hours a day on average, using the NovoTTF-100L(P) System. Application will be continuous for at least 18 hours a day on average.
AND
THERASPHERE® microbeads 120Gy
AND
Systemic therapy:
Physician's choice from either:
(1) Regorafenib (STIVARGA) (160 mg orally once a day, taken days 1-21 of each 28 days cycle) OR
(2) Trifluridine/tipiracil (LONSURF®) 35 mg/m² PO BID on Days 1-5 and Days 8-12 of each 28-day cycle

### Treatment Arm I (Control Arm)

THERASPHERE® microbeads 120Gy
AND
Systemic therapy:
Physician's choice from either:
(1) Regorafenib (STIVARGA) (160 mg orally once a day, taken days 1-21 of each 28 days cycle) OR
(2) Trifluridine/tipiracil (LONSURF®) 35 mg/m² PO BID on Days 1-5 and Days 8-12 of each 28-day cycle.

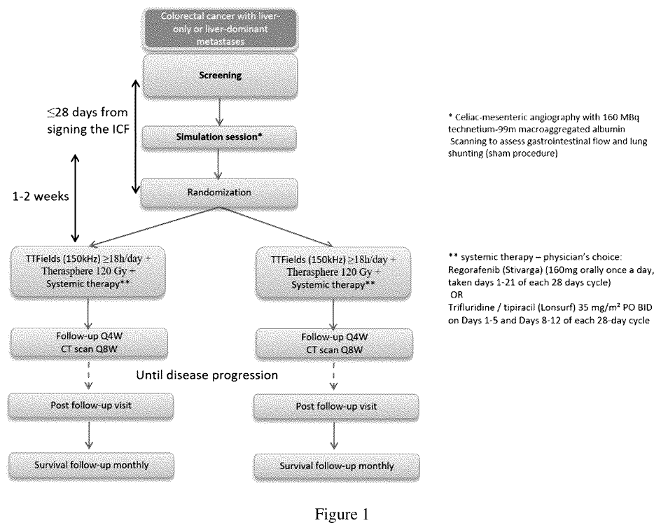

FIG. 1 provides an exemplary study detailing a method of treating colorectal cancer with liver-only or liver dominant metastases.

Number of study patients and statistical considerations: A one-sided logrank test with an overall sample size of 100 subjects (50 in the control group and 50 in the treatment group, including drop out rates of 12%) achieves 79.0% power at a 0.150 significance level to detect a hazard ratio of 0.6548 when the control group median survival time is 5.50.

### Inclusion/Exclusion Criteria

### Inclusion Criteria

≥18 years of age
Histologically confirmed metastatic adenocarcinoma of the colon or rectum, with liver-only or liver-dominant metastases. Patients with liver-dominant metastases are defined as having additional limited extra-hepatic metastases in the lung or lymph nodes (fewer than 5 nodules <1 cm diameter or a single nodule <1.7 cm diameter in the lung, and lymph node involvement in a single anatomical area <2 cm diameter)
Measurable computed tomography (CT) scan evidence of liver metastases which are not treatable by surgical resection or local ablation with curative intent at the time of study entry.
Patients who have been previously treated with or are not candidates for fluorouracil, oxaliplatin, irinotecan, and if Kras wild-type, anti EGFR therapy.
Considered an appropriate candidate for regorafenib or trifluridine/tipiracil therapy.
Measurable disease as measured by Response Evaluation Criteria in Solid Tumors (RECIST) version 1.1.
Eastern Cooperative Oncology Group (ECOG) Performance Status (PS) of 0-1.
Life expectancy ≥3 months.
Ability to understand the nature of this study and give written informed consent.
At least 4 weeks since major surgery
Able to operate the Novo-TTF200T system independently or with the help of a caregiver.

### Exclusion Criteria

Most recent chemotherapy ≤14 days and >Grade 1 chemotherapy-related side effects, with the exception of alopecia.
Treatment with investigational therapy within 28 days prior to initiation of study treatment.
Evidence of potential delivery of greater than 16.5 mCi (30 Gy absorbed dose) per treatment of radiation to the lungs
Evidence of any detectable Tc-99m MAA flow to the stomach or duodenum, after application of established angiographic techniques to stop or mitigate such flow (eg, placing catheter distal to gastric vessels)
Wide field radiotherapy (including therapeutic radioisotopes such as strontium 89) administered <28 days or limited field radiation for palliation <7 days prior to starting study treatment or has not recovered from side effects of such therapy.
Previous radiation delivered to the upper abdomen.
Previously untreated brain metastases. Patients who have received radiation or surgery for brain metastases are eligible if therapy was completed at least 2 weeks previously and there is no evidence of central nervous system disease progression, mild neurologic symptoms, and no requirement for chronic corticosteroid therapy.
Leptomeningeal metastases or spinal cord compression due to disease.
Evidence of ascites, cirrhosis, portal hypertension, or thrombosis as determined by clinical or radiologic assessment.
History of abdominal fistula or gastrointestinal perforation <6 months prior to beginning study treatment.
Serious non-healing wound, active ulcer, or untreated bone fracture.
Presence of active gastrointestinal disease or other condition that will interfere significantly with the absorption, distribution, metabolism, or excretion of oral therapy (e.g., ulcerative disease, uncontrolled nausea, vomiting, diarrhea Grade ≥2, and malabsorption syndrome).
Serious active infection at the time of treatment, or another serious underlying medical condition that would impair the ability of the patient to receive protocol treatment.
Presence of other active cancers, or history of treatment for invasive cancer ≤5 years. Patients with Stage I cancer who have received definitive local treatment and are considered unlikely to recur are eligible. All patients with previously treated in situ carcinoma (i.e., non-invasive) are eligible, as are patients with history of non-melanoma skin cancer.
Use of strong inducers or inhibitors of CYP34A.
The herbal medications St. John's wort, kava, ephedra (ma huang), gingko biloba, dehydroepiandrosterone (DHEA), yohimbe, saw palmetto, and ginseng will not be allowed during study treatment. Patients should stop using these herbal medications 7 days prior to first dose of study treatment.

### Serious Co-Morbidities

a) Hematological, hepatic and renal dysfunction, defined as: Absolute neutrophil count (ANC)<1500/μL, Platelets>75,000/μL, Hemoglobin (HgB)>9 g/dL (pre-enrollment transfusion allowed) White blood cell count<1.5×109/L., ALT and AST>2.5×the upper limit of normal (ULN) or>5 ULN if due to hepatic metastases, total bilirubin>1.5×ULN (unless the patient has grade 1 bilirubin elevation due to Gilbert's disease or a similar syndrome involving slow conjugation of bilirubin), serum creatinine>1.5 mg/dl (133 μmol/L) OR calculated creatinine clearance<50 mL/min.
b) History of significant cardiovascular disease unless the disease is well controlled; significant cardiac disease includes second/third degree heart block; significant ischemic heart disease; poorly controlled hypertension; congestive heart failure of the New York Heart Association (NYHA) Class II or worse (slight limitation of physical activity; comfortable at rest, but ordinary activity results in fatigue, palpitation or dyspnea).
c) History of arrhythmia that is symptomatic or not adequately controlled. Specifically, patients with atrial fibrillation or flutter controlled by medication are not excluded from participating in the trial.
d) History of cerebrovascular accident (CVA) within 6 months prior to randomization or that is not stable.
e) Active infection or serious underlying medical condition that would impair the ability of the patient to receive protocol therapy.
f) Unable to follow the protocol for medical, psychological, familial, geographic or other reasons, or comply with the requirements of the study or to provide consent.
Pregnancy or breastfeeding. Male patients with female partners of childbearing potential and female patients of childbearing potential are required to use two forms of acceptable contraception, including one barrier method, during their participation in the study and for 30 days following last dose. Male patients must also refrain from donating sperm during their participation in the study.
Implanted pacemaker, defibrillator or other electrical medical devices
Known allergies to medical adhesives or hydrogel
Admitted to an institution by administrative or court order

### References

1. Siegel R L, Miller K D, Jemal A. Cancer statistics, 2019. https://doi.org/10.3322/caac.21551. CA: A Cancer Journal for Clinicians. 2019 Jan. 1 2019; 69(1):7-34. doi:https://doi.org/10.3322/caac.21551.
2. Weiss L, Grundmann E, Torhorst J, et al. Haematogenous metastastic patterns in colonic carcinoma: An analysis of 1541 necropsies. https://doi.org/10.1002/path.1711500308. The Journal of Pathology. 1986Nov. 1 1986; 150(3):195-203. doi:https://doi.org/10.1002/path.1711500308.
3. Kelly C M, Kemeny N E. Liver-directed therapy in metastatic colorectal cancer. Expert Review of Anticancer Therapy. 2017 Aug. 3 2017;17(8):745-758.doi:10.1080/14737140.2017.1345629.
4. Wang D S, Louie J D, Sze D Y. Evidence-Based Integration of Yttrium- 90 Radioembolization in the Contemporary Management of Hepatic Metastases from Colorectal Cancer. Tech Vasc Interv Radiol. June 2019; 22(2):74-80. doi:10.1053/j.tvir.2019.02.007.
5. Arnold D, Prager G W, Quintela A, et al. Beyond second-line therapy in patients with metastatic colorectal cancer: a systematic review. Ann Oncol. Apr. 1 2018;29(4):835-856. doi:10.1093/annonc/mdy038.
6. Hendlisz A, Van den Eynde M, Peeters M, et al. Phase III trial comparing protracted intravenous fluorouracil infusion alone or with yttrium-90 resin microspheres radioembolization for liver-limited metastatic colorectal cancer refractory to standard chemotherapy. J Clin Oncol. Aug. 10 2010;28(23):3687-94. doi:10.1200/jco.2010.28.5643.
7. Giladi M, Schneiderman R S, Voloshin T, et al. Mitotic Spindle Disruption by Alternating Electric Fields Leads to Improper Chromosome Segregation and Mitotic Catastrophe in Cancer Cells. Scientific Reports. 2015 Dec. 11 2015;5(1):18046. doi:10.1038/srep18046.
8. Kirson E D, Gurvich Z, Schneiderman R, et al. Disruption of cancer cell replication by alternating electric fields. Cancer Res. May 1 2004;64(9):3288-95. doi:10.1158/0008-5472.can-04-0083.
9. Kirson E D, Dbalý V, Tovaryš F, et al. Alternating electric fields arrest cell proliferation in animal tumor models and human brain tumors. Proceedings of the National Academy of Sciences. 2007;104(24):10152. doi:10.1073/pnas.0702916104.
10. Rominiyi O, Vanderlinden A, Clenton S J, Bridgewater C, Al-Tamimi Y, Collis S J. Tumour treating fields therapy for glioblastoma: current advances and future directions. British Journal of Cancer. 2020 Nov. 4 2020;doi:10.1038/s41416-20-01136-5.
11. Giladi M, Voloshin T, Shteingauz A, et al. Alternating electric fields (TTFields) induce immunogenic cell death resulting in enhanced antitumor efficacy when combined with anti-PD-1 therapy. The Journal of Immunology. 2016;196(1 Supplement): 75.26.
12. Silginer M, Weller M, Stupp R, Roth P. Biological activity of tumor-treating fields in preclinical glioma models. Cell Death & Disease. 2017 Apr. 1 2017;8(4):e2753-e2753.doi:10.1038/cddis.2017.171.
13. Gotlib K, Zeevi E, Schneiderman R S, et al. Abstract 1865: The combined treatment of 150 kHz Tumor Treating Fields (TTFields) and sorafenib inhibits hepatocellular carcinoma in vitro. Cancer Research. 2018;78(13 Supplement):1865. doi:10.1158/1538-7445.AM2018-1865.
14. Grosu A, Gkika E, Brunner T, Thimme R, Weinberg U. Abstract CT175: HEPANOVA Phase II study design for advanced hepatocellular carcinoma: Tumor treating fields concomitant with sorafenib. 2019:CT175-CT175.
15. Valderrama-Treviño et. al., Hepatic Metastasis from Colorectal Cancer, Euroasian J Hepatogastroenterol, July-December 2017;7(2):166-175.

Patents and technical literature cited herein are incorporated by reference in their entirety in the specific context indicated.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

1.-21. (canceled)

22. A method of treating cancer in a subject, comprising:
delivering radioactive particles to an organ of the subject, wherein the organ contains at least one cancer cell;
applying alternating electric fields to the organ at a frequency of 50 kHz to 10 MHz, wherein the method comprises applying alternating electric fields prior to delivering the radioactive particles; and
administering systemic cancer therapy to the subject.

23. The method of claim 22, wherein the cancer is colorectal cancer that has metastasized to a liver of the subject, and wherein the radioactive particles are delivered to the liver.

24. The method of claim 22, wherein the alternating electric field has a frequency between 100 and 300 kHz.

25. The method of claim 22, wherein the radioactive particles comprise at least one of resin-based microspheres and glass-based microspheres.

26. The method of claim 22, wherein at least a portion of the applying step is performed simultaneously with at least a portion of the delivering step.

27. The method of claim 22, wherein the alternating electric fields are applied for at least 18 hours.

28. The method of claim 22, wherein the organ is located in an abdomen or a head of the subject.

29. The method of claim 28, wherein the organ is a liver, a spleen, or a pancreas.

30. The method of claim 22, wherein the radioactive particles comprise Yttrium-90, Cesium-131, Gold-198, Iridium-192, Iodine-125, Indium-111, or Gallium-68.

31. The method of claim 30, wherein the radioactive particles comprise Yttrium-90.

32. The method of claim 22, wherein the systemic cancer therapy comprises administering a chemotherapeutic agent to the subject.

33. The method of claim 32, wherein the chemotherapeutic agent comprises at least one of regorafenib and trifluridine.

34. A method of preventing recurrence of cancer in a subject that has been previously treated for cancer comprising applying alternating electric fields to an organ of the subject at a frequency of 50 kHz to 10 MHz for a period of at least three months, wherein the previously treated cancer is a primary cancer or a metastatic cancer.

35. The method of claim 34, wherein the frequency of the alternating electric fields is from 100 kHz to 300 kHz.

36. The method of claim 34, wherein the organ is located in an abdomen or a head of the subject.

37. The method of claim 34, wherein the organ is a liver, a spleen, or a pancreas.

38. The method of claim 34, further comprising delivering radioactive particles to the organ.

39. The method of claim 38, wherein the radioactive particles comprise at least one of resin-based microspheres and glass-based microspheres.

40. The method of claim 34, further comprising administering systemic cancer therapy to the subject.

41. The method of claim 40, wherein the systemic cancer therapy comprises administering to the subject a chemotherapeutic agent.

42. The method of claim 22, wherein the intensity of the alternating electric fields is 0.1 to 20 V/cm (RMS) for at least a portion of a region to which the alternating electric fields are applied.

43. The method of claim 42, wherein the intensity is 0.5 to 10 V/cm (RMS).

44. The method of claim 42, wherein the intensity is 1.0 to 2.5 V/cm (RMS).

45. The method of claim 24, wherein the alternating electric field has a frequency between 120 kHz to 180 KHz.

46. The method of claim 24, wherein the alternating electric field has a frequency of 150 kHz.

47. The method of claim 35, wherein the alternating electric field has a frequency between 120 kHz to 180 kHz.

48. The method of claim 35, wherein the alternating electric field has a frequency of 150 kHz.

49. The method of claim 41, wherein the chemotherapeutic agent is one or more of regorafenib and trifluridine.
